(19) United States
(12) Patent Application Publication
(10) Pub. No.: US 2003/0203854 A1
(43) Pub. Date: Oct. 30, 2003

(54) Composition for effecting serum cholesterol levels

(76) Inventors: Ivo Pischel, Trostberg (DE); Herbert Clinton Fairow, Tolone, IL (US); Ralf Jager, Champaign, IL (US)

Correspondence Address: FULBRIGHT & JAWORSKI, LLP, 666 FIFTH AVE, NEW YORK, NY, 10103-3198, US

(21) Appl. No.: 10/131,620

(22) Filed: Apr. 23, 2002

(51) Int. Cl. A61K 31/7024; A61K 31/202; A61K 31/16; A61K 31/225

(52) U.S. Cl. 514/23; 514/560; 514/625; 514/561; 514/547

(57) ABSTRACT

A new composition for effecting serum cholesterol levels is claimed, said composition comprising a) at least a waxy acid with 23 to 50 carbon atoms or derivatives thereof and with serum cholesterol level reducing properties, and b) 0 to 99.99% by weight of at least a component with serum cholesterol level effecting properties, and c) 0 to 20% by weight of at least a pharmaceutically acceptable formulation aid. The efficacy of this composition can be enhanced by further incorporation of other cholesterol reducing agents, like lecithin, tocotrienol, saponins, fibers, long-chain waxy alcohols and niacin. The claimed composition combines the benefits of the single physiolocology active constituence together with the technology to increase the biovailability.

### FIELD OF THE INVENTION

The present invention relates generally to a composition for effecting serum cholesterol levels and, more particularly, to a composition for reducing serum cholesterol comprising waxy acids or waxy acids and (phyto)sterols and/or stanol ideally in a highly biovailable formulation like micelles or liposomes. The efficacy of this composition can be enhanced by further incorporation of other cholesterol reducing agents, like lecithin, tocotrienol, saponins, fibers, long-chain waxy alcohols and niacin.

### BACKGROUND OF THE INVENTION

Elevated serum cholesterol levels (>200 mg/dL) have been indicated as a major risk factor for heart disease, the leading cause of death. As a result, experts have recommended that those individuals at high risk decrease serum cholesterol levels through dietary changes, a program of physical exercise, and lifestyle changes. It is recommended that the intake of saturated fat and dietary cholesterol be strictly limited and that soluble fiber consumption be increased. Strictly limiting the intake of saturated fat and cholesterol does not, itself, present a risk to proper health and nutrition. Even where saturated fat and cholesterol are severely restricted from the diet, the liver remains able to synthesize sufficient quantities of cholesterol to perform necessary bodily functions.

More recently, experts have begun to examine the individual components of the lipid profile, in addition to the total cholesterol level (TC). While an elevated TC is a risk factor, the levels of the various forms of cholesterol which make up TC may also be risk factors. Elevated low-density lipoprotein (LDL) is a cause for concern, as these loosely packed lipoproteins are more likely to lodge within the cardiovascular system leading to the formation of plaque. Low levels of high-density lipoproteins (HDL) are an additional risk factor, as they serve to sweep artery-clogging cholesterol from the blood stream. A better indication of risk appears to be the ratio of TC:HDL.

A number of nutritional factors have been shown to improve serum cholesterol levels. For example, the use of phytosterols and—stanol and their esters has been well documented in human clinical trials and in animal studies to lower serum cholesterol levels. This cholesterol lowering effect has been attributed to interference with the absorption of dietary cholesterol. Phytosterols, being structurally similar to cholesterol, competitively bind with cholesterol sterol receptor sites, thus preventing cholesterol uptake. Unlike their cholesterol counterparts, phytosterols are very poorly absorbed, and some are not absorbed at all. Therefore, phytosterols do not contribute to an increase in serum cholesterol levels. In addition to competing for receptor sites, phytosterols also compete for the enzyme cholesterol esterase. This enzyme is required by cholesterol for its breakdown to components, which may be absorbed through the microvilli which line the wall of the small intestine. Thus, phytosterols also impede the enzymatic breakdown and intestinal absorption of cholesterol, which further reduces serum cholesterol levels. A newer group of sterols that functions similar to phytosterols are the phytostanols, which also naturally occur in small quantities in plant, and can be easily obtained by catalytic reduction of the corresponding phytosterols.

Plant derived long-chained aliphatic alcohols have also been documented to reduce serum cholesterol levels in experimental models, healthy humans and in type II hypercholesterolemic patients. These aliphatic alcohols with chain lengths of C24-C36, collectively known as policosanol, have been employed in the treatment of elevated serum cholesterol levels in only the past five years, but policosanol has shown much promise, as reported in a number of published human clinical trials. The mechanism of action has not yet been elucidated, but policosanol's effectiveness is attributed to its influence on the biosynthesis of cholesterol within the liver. This accounts for the ability of policosanol to not only decrease total cholesterol, but also to decrease LDL serum levels and increase HDL levels.

Both phytosterol/stanol as well as waxy acids are water insoluble and therefore show low bioavailability. U.S. Pat. No. 5,502,045 describes a method for reduction of serum cholesterol with sitostanol-ester dissolved in dietary fat like margarine. However, this invention shows the disadvantage of requiring the administration of 23-50 g/day of dietary fat and of being less effective at reducing cholesterol absorption in humans compared to the unesterified sterol. The only benefit by using the ester form of the stanols in their solubility in fats, which helps dispersing them in the gastro-intestinal tracts after ingestion.

U.S. Pat. No. 5,244,887 comprises the use of stanols including sitostanol in food additives to reduce cholesterol absorption. In U.S. Pat. No. 5,244,887, for an additive preparation sitostanol is dissolved with an edible solubilizing agent such as triglyceride, an antioxidant such as tocopherol, and a dispersant such as lecithin, polysorbate 80, or sodium lauryl sulfate. U.S. Pat. No. 5,118,671 teaches the production of sitosterol-lecithin complexes for pharmaceutical use but does not mention its oral dosage for cholesterol lowering. U.S. Pat. No. 6,063,776 discloses a method to form micellar phytostanols, which leads to a high bioavailability of the emulsified active ingredients. The main emulsifiers used are lecithin or sodium stearoyl-2-lactylate.

### SUMMARY OF THE INVENTION

The present invention provides a composition for effecting serum cholesterol levels in humans and animals. The composition comprises a) long-chain waxy acids and b) up to about 99.99% by weight of a component with serum cholesterol level effecting properties, like phytosterol. The composition further comprises as third component c) from 0% to about 20% by weight of a pharmaceutically acceptable formulation aid, such as diluents, stabilizers, binders, buffers, lubricants and aromatics, coating agents, preservatives, emulsifiers and suspension agents and surface active compounds.

In a preferred embodiment, the claimed composition comprises from about 20% to about 90% by weight of phytosterol, and from about 1.0% to about 40% by weight of long-chain waxy acids. In the most preferred embodiment of the invention, the composition comprises about 100:1 parts by weight of phytosterol and long-chain waxy acids.

### DETAILED DESCRIPTION OF THE INVENTION

As noted previously, “long-chain fatty acids” means within this invention a mixture of high-molecular weight carbon acids having chains from C23 to C50 (Table 1). These fatty acids occur naturally in wax form. They can be comprised of straight or branch chains, and of saturated or unsaturated nature. (Very) long-chain fatty acids are isolated from a number of different plant or animal sources, including for instance sugar cane wax, rice bran wax, beeswax, wool wax, and shellac wax. The processes to obtain the waxy acids are state-of-the-art and the procedures are well described, e.g. in the Chapter “Waxes” of Ullmann's Encyclopedia of technical chemistry, 3^{rd} or 4^{th} Edition, or Kirk-Othmer's Encyclopedia of Chemical Technology, 3^{rd} ed.

Furthermore synthetic of fossil waxy acids can also be used within this invention. For instance, waxy acid derived by catalytic oxidation of paraffins, polyethylenes, or long-chain omega-olefins, like oxidized Unilin (synthetic waxy alcohols), or Unilox (synthetic waxy acids) of Baker Petrolite, or montan-wax derived acid waxes, e.g. Licowax S, or Licowax WS of Clariant, German.

The composition of the waxy acids depends on the kind and origin of the waxes used, but mainly they comprise long-chain waxy acid with chain-lengths of C24 through C36, whereby the number of carbon atoms in the chain is even.

There are preferably used within the scope of this invention as waxy acid component a) an anhydride, a substituted or unsubstituted amid, a salt of ammonium, a salt of an alkaline or earth alkaline, a salt of an amino acid, a salt of an organic base such as a substituted ammonium, an ester of a mono-, di- or tri-alcohol, an ester of an oligo- or polyalcohol with 1 to 10 carbon atoms, an ester with a phyto-compound such as phytosterole, a sugar or a mono-, di- or triglyceride, whereby this component may be of biological, geological or synthetic origin and is preferably derived from plant, animal or mineral oils and/or waxes.

This invention also comprises a composition with a hydrolysed waxy acid ester as waxy acid component; this waxy acid ester can preferably contain waxy alcohols, that show cholesterol effecting properties, which means properties, that control the cholesterol absorption or synthesis or that support the cholesterol excretion.

TABLE 1
Percentage of the different waxy acids of the waxy acid fraction (Ullmann)

| Wax | | | | |
|---|---|---|---|---|
| [%] of acid | Carnauba | Montan | Candelilla | Beeswax |
| 14.1 | 21 | 7 | 1 | 47 |
| C < 24 | | | | |
| C 24 | 23 | 8 | 1 | 10 |
| C 26 | 11 | 13 | 1 | 4 |
| C 28 | 16 | 18 | 3 | 4 |
| C 30 | 6 | 18 | 36 | 3 |
| C 32 | 2 | 7 | 49 | 2 |
| C 34 | 1 | 3 | 8 | 1 |
| C 36 | 0 | 1 | 1 | |
| C > 36 | 0 | 1 | | |

Other wax suiting as raw materials are spermaceti, shellac, Japan, ouricury, and/or rice branwax.

This selection should serve just as examples and should not limit the present invention.

Phytosterols are mixtures of waxy plant sterols like beta-sitosterol, stigmasterol, and campesterol. They are naturally occurring in many common vegetable food products. The particular phytosterol used in the preferred embodiment of the invention is derived from vegetable oil and has the formulation set forth in Table 2. This material is sold under the trademark CHOLESTATIN® and is available from Degussa BioActives TracoLabs, Inc. Again, however, it should be understood that the invention is not limited to this particular phytosterol product, and that any number of other commonly available phytosterols can also be used.

TABLE 2
Phytosterol composition

| Component | Content | CHOLESTATIN ® |
|---|---|---|
| [0018] Total sterols | ˜85% | — |
| Beta-Sitosterol. | ˜45% | 40% Min |
| Campesterol | ˜25% | 20% Min |
| Stigmasterol | ˜15% | 11% Min |

As previously mentioned, the current invention differs from prior art compositions of plant sterols and sitostanol as well as waxy acids in many significant ways.

As component b) of the claimed composition are preferably used components with cholesterol-absorption controlling properties and/or properties that control the cholesterol synthesis and/or that support cholesterol excretion. Suitable in this connection are sterol compounds of plant origin and more preferably phytosterols, phytostanols and acceptable derivatives or mixtures thereof, because of their properties of controlling the cholesterol absorption. The besaid sterol component b) should be a branched or non-branched and/or (un-)saturated fatty acid ester and/or can be derived from a vegetable oil or by synthesis therefrom.

Preferred components b) are also policosanoles or statines, because they may control cholesterol synthesis. Polysaccharids like pectine are suitable because of supporting cholesterol excretion.

First, the now combined use of phytosterols and waxy acids enables a more effective, two pronged approach to lower blood cholesterol than the single components do. Second, the preferred compositions do not necessarily contain triglycerides, fats, or oils. The phytosterol and waxy acids need not be dissolved in fat, but rather are combined with an emulsifier to form an aqueous vesicular complex, which can enter directly into the intestinal micelle phase. Third, the mixture can be prepared in solid form by drying aqueous or solvent micellar formulations of phytosterol/waxy acid/emulsifier. Forth, the combination can be added to non-cholesterol-containing and fat-free foods and beverages. Fifth, the mixture is prepared in a manner to prevent self-association of components as occurs when it is dried from organic solvents containing sitostanol and solubilizing agents. The composition herein referenced has the advantage of a high degree of bioavailability as assayed with artificial bile in vitro. This is significant and cannot be achieved with fat carrier systems.

The claimed composition is useful for controlling and reducing cholesterol absorption in humans at dosages per day between 10 and 1500 mg, and a preferred dose is 500-1500 mg. These doses are less than required by current protocols. The composition may be used in capsule, tablet, barr, beverage, oil, oil- of fat based emulsion, milk based product or suspension from, as a drug or dietary supplement. Alternatively, it may be used in foods as a food additive or substance generally recognized as safe for human consumption, whereby a small partical form, and hereby a liposome or micellar form is preferred.

In preparation of the composition useful for reducing serum cholesterol in highly bioavailable form, the first step is to provide an aqueous homogeneous micellar mixture for the plant sterol with the preferred emulsifier of choice.

The preferred method is to use a combination of phytosterols and waxy acids because only small amounts are absorbed in the small intestine, but on the other hand, this plant sterol shows high inhibition properties of cholesterol absorption. Similar phytosterol compounds are also suitable, including sitosterol, campesterol, campestanol and stigmasterol.

Preferred is also a composition that comprises 1 to 40% by weight of the waxy acid component a) and 20 to 90% by weight of component b) The weight-to-weight-ratio of the waxy acid component and component b) shall be within the range of 1:10 to 100 and preferably 1:99. Additionally, there is claimed a composition with a waxy acid component a) that acts as an additional emulsifier.

Emulsifiers usually used are proteins and phospholipids such as lecithin, and the most preferred phospholipid systems useful to enhance the bioavailability is a mixture of lecithin and related products. Also effective agents used of this invention are SSL, bile acids, saponins, glycolipids and soaps of fatty- and waxy acids. The advantage of using saponins and waxy acid soaps are their property to lower themselves cholesterol, so they are excipient and active ingredient in one.

Synthetic emulsifiers such as mono- or diglycerides, esters at acetic, citric, lactic and tartaric acid, stearoyllactylate and salts hereof as well as sugar esters and (poly-)sorbitans are also useful as emulsifiers in producing the composition.

The way of preparation is to provide a fine emulsion of plant sterols and waxy acids in aqueous homogeneous mixture by using the above mentioned emulsifiers. The emulsion can be produced by well established methods employing high shear mixing, like vortexing, sonicating, passing through a small orifice, and similar processes.

Another process to generate highly dispersed micellar vesicles is to drop a solution of the mixture of the active ingredients and the emulsifier in an aqueous media in combination with agitation and reduced pressure to eliminate the solvent. The drying process can be vacuum drying, freeze drying or low-temperature ambient air drying at low temperature.

For a production natural phytosterol mixtures (e.g. Cholestatin®) and waxy acid mixtures (e.g.DWAC#1) in the preferred ratio 100:1 were mixed together in different solvents at various ratios of 1:1 through 1:5 by weight. The mixtures were then transferred to an evacuation tube and the solvent was removed under slightly reduced pressure and warming up. Solvents used for this purposes have been unpolar ones like pentane, hexane or petrolethers, alcohols like methanol, ethanol, iso-propanol and higher ones, esters like ethyl acetate, ethers, ketone, and mixtures of them. Thereafter, the dry materials were transferred into water and sonicated to build a stable suspension. The stability of the suspension and the micelles were evaluated by turbidity measurements using the nephelometric principle like the US EPA 180.1 method. Microscopic methods or a Zetasizer can be used to evaluate the integrity and size of the micelles as well. The obtained stable suspension can be dried using careful drying methods like freeze-drying, lyophilization, or low temperature water-evaporation. The so produced materials are easily re-suspended in aqueous media to produce highly dispersed formulations.

As earlier mentioned, the daily dosage of the dry powder should be within the range of 100 to 2000 mg, and preferred 250 to 1000 mg per day. This daily dose should preferably be applied in divided portions, one to four times daily.

As noted above, phytosterol and (very)long-chain fatty acids lower serum cholesterol by two independent and unrelated mechanisms of action. Both compounds together are expected to have a synergistic effect on lowering serum cholesterol. As previously mentioned, phytosterols impede the enzymatic breakdown and intestinal absorption of cholesterol, which reduces serum cholesterol levels. (Very)long-chain fatty acids act directly on the cholesterol synthesis pathway itself, thereby inhibiting the biosynthesis of cholesterol from saturated fat. The combination of phytosterol and (very)long-chain fatty acids into a single composition is expected to provide a more effective treatment for elevated serum cholesterol than would be expected from the additive effect of both components.

The composition according to this invention can also comprise as additional component c) a formulation aid that is one of the group consisting of fillers, stabilizers such as antioxidants, diluents, binders, buffers, lubricants and aromatics, preservatives, emulsifiers, coating agents, suspending agents and surface active compounds. This composition can also comprise additionally other components with serum cholesterol effecting properties such as tocotrienols, saponins, fibers, long-chain waxy alcohols of 20 to 50 carbon atoms and their acceptable derivatives, artichoke-extracts and alfalfa-extracts, statins, bile sequestrants or niacin. There is also claimed a composition with a weight-to-weight ratio of the components a):b):c) that is 1:79:20.

One major point of the present invention are phytosterol-waxy acid esters, which are not described yet and their synthesis. These esters are structurally similar to the short chain fatty acids of phytosterol of the U.S. Pat. No. 5,502,045, but the efficacy of the new chemical entities is beyond the embodiment of U.S. Pat. No. 5,502,045, because of the combinatorial approach of two physiologically active building blocks. Similar to the esters disclosed in U.S. Pat. No. 5,502,045 the claimed phytosterol-waxy-acid-esters that are cleaved by the intestinal juice, particularly by pancreatic lipases to release the cholesterol lowering phytosterol or waxy acids in highly dispersed micellar or liposomal form to become highly effective. The cleavage of waxy esters are described in the literature, e.g. Place A R; Am J Physiol September 1992:263 (3 Pt 2):R464-71), which show the efficacy of cleavage waxy ester of about 50% in mammals. Fishes and birds are able to go even beyond 50% in cleavage.

Preferably there are claimed esters of phytosterol-waxy-acids that contain as phytosterol component a branched or non-branched and/or saturated or unsaturated fatty-acid ester and/or, wherein the waxy acid component, preferably with 23 to 50 carbon atoms, is of vegetable or lignit origin.

Previous clinical and toxicological testing of waxy acids and phytosterol has shown that the tolerance of both components is good. Phytosterols occur in almost all plant-derived foodstuff. The estimated daily intake by ingestion of a balanced diet in the Western Countries is usually over 250 mg (Hicks K B, Moreau R A, Food Technol, Vol 55, No 1, January 2001). Waxy acids are also found regularly in food material (Kawahara K et al. J Nutr Sci Vitaminol (Tokyo) December 1988; 34(6):633-9). Toxicological data on semi-synthetically derived waxy acids are public (Lange J, Wildgruber J; Fette, Seifen, Anstrichmittel, Jahrgang 78, Nr. 2, 1976; Gamez R, Toxicol Letters Dec. 20, 2000; 118(1-2):31-41).

The herein-disclosed composition combines the benefits of the single physiologically active constituents together with the technology to increase the bioavailability. Additionally, the invention discloses formulations, whereas for instance the emulsifier is not only an adjuvant, but also an active component. Thus, the purpose to favorite lecithin and/or saponins as emulsifier is not only because of their activity as surfactant, but also as cholesterol-lowering agent according to Dewailly P et al.; Effects of Polyenylphosphatidylcholine on Lipoproteins in Patients with Hypercholesterinemia, Die Medizinische Welt 12/85. Therefore, in opposite to U.S. Pat. No. 6,063,776 the use of lecithin should not be limited, but have to be present in a sufficient amount to be a useful addition to the formulation as emulsifier and as an active agent.

The following examples are offered to further illustrate, but not limit the process of the present invention.

### EXAMPLES OF PREFERRED EMBODIMENTS

In these first three examples formulations to produce tablets or capsules are described which comprise combinations of phytosterol and waxy acids. The next examples disclose the procedures to prepare micellar formulations. Whereas the following example describes the efficacy in an animal model.

Phytosterols, as used here, mean in the most cases the natural occurring mixtures of sterols such as sitostanol, sitosterol, campesterol, stigmasterol, and their derivatives and reduction products. Phospholipids, as used here, mean glycerophospholipids and sphingolipids, as well as their derivatives, such as lysophospholipids. Saponins, as used here, in most cases mean soy saponins obtained during the soy bean utilization process.

Examples of compositions made according to the invention is set forth below:

### Example 1

Tablet Formula:
Composition

| Ingredient | Amount/tablet | | Function |
|---|---|---|---|
| Phytosterol complex (>88%) | 250 | mg | active |
| (e.g. Cholestatin ®) | | | |
| Waxy acids | 5 | mg | active |
| Calcium phosphate | 261.7 | mg | excipient |
| Cellulose | 49.4 | mg | excipient |
| Silicon dioxide | 100 | mg | diluent |

The preparation of the blend is accomplished by thoroughly mixing of the above ingredients using a lab powder bender. The obtained blend is pressed to tablets using a bench tablet press.

### Example 2

Formula for Chewable Tablets

| Ingredient | Amount/tablet | | Function |
|---|---|---|---|
| Phytosterol complex (>88%) | 430 | mg | active |
| (e.g. Cholestatin ®) | | | |
| Waxy acids | 10 | mg | active |
| Dextrose | 700 | mg | sugar |
| Sorbitol | 75 | mg | sweetener |
| Natural a/o Artificial flavors | 50 | mg | excipient |
| Stearic acid | 25 | mg | excipient |
| Silicon dioxide | 10 | mg | excipient |
| Magnesium Stearate | 4 | mg | excipient |
| Succinic acid | 3 | mg | excipient |
| Food color blend (var) | 2 | mg | Coloring |

The preparation of the blend is accomplished by thoroughly mixing of the above ingredients using a lab powder bender. The obtained blend is pressed to tablets using a bench tablet press.

### Example 3

Soft Gelatin Capsule Formulation

| Ingredient | Amount/Capsule | | Function |
|---|---|---|---|
| Phytosterol complex (>88%) | 250 | mg | active |
| (e.g. Cholestatin ®) | | | |
| Waxy acids | 5 | mg | active |
| Lecithin | 50 | mg | active/processing |
| aid | | | |
| MCT (or substitute) | 445 | mg | diluent |

The preparation of the blend is accomplished through suspending the phytosterol complex, waxy acids, lecithin and any other desired components as described previously, in the carrier oil (MCT or any acceptable substitute). Further processing may include milling to ensure an encapsulatable particle size. Capsule fill weights from 500 mg to 1400 mg are envisioned to supply various quantities of active components. This blend is encapsulated at a toll encapsulator.

### Example 4

Procedure A to Prepare Micellar/Liposomal Formulations Using Soy Saponins

1 gram Phytosterol complex (e.g. Cholestatin®) and 15 mg of waxy acid complex (e.g. DWAC#1) are boiled in 50 ml of ethyl alcohol with 1.0 gram of a 20% soy saponin mixture. The solution is dropped into a rotary evaporator vessel containing 250 mL vigorously stirred water under a vacuum of 100 mbar pressure, and at 50° C., which supports the evaporation of the alcohol. Under these conditions a dispersion containing fine particles is obtained.

### Example 5

Procedure A to Prepare Micellar/Liposomal Formulations Using Lecithin

1 gram Phytosterol complex (e.g. Cholestatin®) and 15 mg of waxy acid complex (e.g. DWAC#1) are boiled in 50 ml of ethyl acetate/iso-propanol mixture together with 1.0 gram of a 20% lecithin (e.g. Epikuron). The solution is dropped into a rotary evaporator vessel containing 250 mL vigorously stirred water under a vacuum of 100 mbar pressure, and at 50° C., which supports the evaporation of the alcohol. Under these conditions a dispersion containing fine particles is obtained.

### Example 6

Procedure B to Prepare Micellar/Liposomal Formulations

To prepare the phytosterol/waxy acid/emulsifier vesicles in a 1:1 to 1:10 mass ratio, 2.5 gm of Cholestatin®, 25 mg of DWAC waxy acids, or instead 25 mg of the reacted Cholestatin®-DWAC of example 7, was added to 2.5 to 25 gm of high phosphatidylcholine containing soy lecithin (e.g. Epikuron 130, or 200) in a 250 mL glass beaker. Different solvents in sufficient amount are added to dissolve the mixture with stirring to solubilize all components, and the solvent was then removed by elevated temperature (max. 75° C.).

After all the solvent was removed, at the end by employing reduced pressure for several hours (6 to 36 h) the solid in the beaker was then broken up with a glass rod, 100 mL of water was added per 10 g of solid, and the suspension was stirred vigorously for one hour. Micelles were prepared by sonicating the contents of the beaker with an ultrasonic bath at ambient temperature for 5 to 1.5 h.

The diameter of the micelles is about 250 nm. The turbidity of the aqueous suspensions is constant over a certain period of time, which indicates their stability.

### Example 7

Preparation of Sterol Esters of Waxy Acids as New Chemical Entities

The preparation of phytosterol ester of very-long-chain fatty or waxy acids can be achieved by reaction between phytosterols and waxy acids (carbon chain higher than C22) or mixtures thereof at 200 C for 1-3 hours. The reaction product can be used as is or after re-crystallization from acetone or other appropriate solvents. The obtained products are soluble in fatty products like margarine or vegetable and other edible oils.

100 mg of the sterols and 100 mg of the waxy acids are mixed thoroughly and heated in a heating block for 2 hours at 200° C. After cooling the reaction product is ground and re-crystallized from 5 ml of acetone. The fine crystalline material is collected by filtration and dried at ambient temperature in the air. The yield in all cases is around 150 mg.

Thereafter, TLC was used to analyze the chemical entities. The melting points significantly show a new type of composition.

Conditions for the TLC:

Stationary Phase: flexible plates for TLC Whatman AL SIL G/UV, 250 um Silica Gel Layer (cat#4420 222)

Mobile Phase: Chloroform/Ethanol 99:1

Development/Detection: 50% Sulfuric acid, heated

Melting Point: Capillary, open, Thiele apparatus

Sterols used:

| | | | | | Rf |
|---|---|---|---|---|---|
| # | Sterol | Manufact. | FW | mp/deg C | (TLC) |
| ST1 | Stigmasterol | Sigma # S-2424 | 412.7 | 165-167 | 0.26 |
| ST2 | Beta-Sitosterol | Aldrich # S340-2 | 414.72 | 139-142 | 0.24 |
| ST3 | Cholestatin ® | Phytosterol | | | 0.25 |
| | | complex | | | |

Wax acids used:

| | | | | | Rf |
|---|---|---|---|---|---|
| # | Wax acid | Manufact. | FW | mp/deg C | (TLC) |
| WA1 | Tetracosanoic | Aldrich # | 368.65 | 75-83 | 0.23 |
| | 99% | 23,468,0 | | | |
| WA2 | Octacosanoic | Aldrich # | 424.76 | 92-94 | 0.21 |
| | 98% | 28,443-2 | | | |
| WA3 | Degussa waxy | Degussa AG, | | | 0.21 |
| | acid complex | DWAC # 1 | | | |
| WA4 | Montanic acids | e.g., Clariant | | | 0.22 |
| Waref | Palmitic | Aldrich # P5-1 | 256.43 | 61-64 | 0.36 |

The following reactions have been conducted:

Product of

#1 ST1+WA1

#2 ST2+WA1

#3 ST3+WA1

#4 ST1+WA2

#5 ST2+WA2

#6 ST3+WA2

#7 ST1+WA3

#8 ST2+WA3

#9 ST3+WA3

#10 ST1+WA4

#11 ST2+WA4

#12 ST3+WA3

#13 ST3+WAref

The compounds shown in the tables above are new chemical entities, and not described in the literature so far.

#1 Stigmasterol lignocerate

#2 beta-Sitosterol lignocerate

#4 Stigmasterol montanate

#5 beta-Sitosterol montanate

All other preparations (#3, 6-12) contain a variety of new components due the nature of the raw materials, which a mixtures of waxy acids, or phytosterols, respectively.

### Example 8

Production of Sterol Esters—Phytosterol Combinations

The production of phytosterol esters of very-long-chain fatty or waxy acids combined with phytosterol in the appropriate ratio can easily be accomplished by heating and melting a blend of a natural phytosterol complex (e.g. Cholestatin®) with a waxy acid complex (e.g. DWAC). Blends of those components in a ratio 100:1 were heated up to 200° C. for 1 to 5 hours. The reaction product can be used as is or after re-crystallisation from acetone or other appropriate solvents. The obtained products are soluble in fatty products like margarine or vegetable and other edible oils.

After processing with emulsifiers, it can be used in beverages or other aqueous media.

100 kg of Cholestatin® and 1 kg of the waxy acid products (Montanic acids or DWAC#1) are mixed thoroughly and heated in a reaction vessel equipped with a stirring 2 hours at 200° C. After cooling the reaction product is ground, and ready to used with the products and formulations described in the present invention. The yield is quantitative. The reaction of Cholestatin® with DWAC#1 or montanic acids leads to a product with physical-chemical properties similar to Cholestatin®. The existence and content of Cholestatin® as well as the reaction product was evaluated by TLC, and saponification number.

The analysis showed a quantitative conversion of the acid-components.

### Example 9

Incorporation of Present Invention into Salad Dressing and Similar Entities.

The present invention has been successfully incorporated into horseradish sauce at a level of 0.65 grams per serving (9 grams). Presence of the invention had no adverse effect on the taste or texture of the horseradish sauce.

In addition, a finely ground form of the present invention was incorporated into a fat free French Dressing (Marzetti's). The presence of the invention in no way adversely affected the taste or texture of the salad dressing.

### Example 10

Animal Efficacy Study

The effect of the waxy acid and phytosterol/waxy acid combination on cholesterol absorption was compared within an animal model under atherogenic diet.

The purpose of the described research was to examine the efficacy and mechanism of action of orally administered long chain fatty acid complexes on cholesterol and lipoprotein profiles, and cholesterol synthesis, in the hamster which has been identified as a good model of human lipid metabolism (Dietschy et a. 1993). The objectives of the conduced investigation were to compare the action of different phytosterol/waxy acid preparations, versus a control preparation without active components, on cholesterol levels in plasma, and de novo cholesterol biosynthesis.

Seventy-two Golden Syrian male hamsters weighing between 100 and 120 g have been used in this experiment. The animals were to be systematically randomized in 6 groups of 12 hamsters per group, housed in individual cages and subjected to 12 hr:12 hr light:dark cycling for 2 week before commencement on the experimental protocol. During the pre-experimental and experimental periods, hamsters will be provided with free access to water, and be fed ad libitum a semi-synthetic diet (control diet), based on the composition of the AIN-76A which contains 0.025% cholesterol. The total fat content of this diet will be 5% fed in the form of a mix of lard and safflower oil to provide a P/S ratio of 0.4. The control diet alone has been be fed to group 1. Groups 2-4 have been supplied with the control diet containing 15 mg/kg of either waxy acids formulations or the phytosterol complex. Group 5 and 6 were provided with the combination of the sole component of the groups 2-4. Food intake and body weight of individual animals will be monitored regularly through the feeding period.

After 30 days on diets, hamsters in each group will be anesthetized with halothane. Blood samples were collected for total cholesterol, low density lipoprotein (LDL) and high density lipoprotein (HDL) subclass cholesterol and triglyceride (TG) levels.

Experimental Method for Lipid Analysis

Plasma total cholesterol, HDL cholesterol and TG levels have been measured using a VG Autoanalyzer in conjunction with commercial enzymatic kits and appropriate standards (Abbott Diagnostics). The concentration of non apolipoprotein (Apo)-A cholesterol, taken as LDL-cholesterol, have been calculated as total cholesterol minus HDL cholesterol concentration. In the case of the hamster it is considered inappropriate to use the Friedewald equation due to the distribution of lipids across lipoprotein groups (Friedewald et al. 1972, Gidez et al. 1992). All samples were processed through the above system in duplicate.

TABLE A
The effect of phytosterol, waxy acids, and their combination on hamster
lipid profiles (% change of control mean)

| Diet | CHOL | LDL |
|---|---|---|
| Control | 0 | 0 |
| DWAC#1 | −8.32 ± 10.14 | −5.53 ± 32.45 |
| Licowax | −8.15 ± 13.46 | −5.63 ± 37.75 |
| Cholestatin ® | −21.24 ± 13.75 | −14.17 ± 40.46 |
| Cholestatin ® PLUS DWAC#1 | −30.1 ± 11.84 | −20.07 ± 35.29 |
| Cholestatin ® PLUS Licowax | −29.13 ± 12.32 | −19.52 ± 32.62 |

This results show that, compared to placebo, waxy acids, phytosterol, and in particular the combination of both are effective in lowering cholesterol blood levels. The mechanism of action of waxy acids seems to be different from that of the phytosterol and explains the synergetic effect.

While preferred embodiments have been shown and described, various modifications and substitutions my be made without departing from the spirit and scope of the invention. Accordingly, it is to be understood that the present invention has been described by way of example and not by limitation.

What is claimed is:

1. A composition for effecting serum cholesterol levels, said composition comprising
at least a waxy acid with 23 to 50 carbon atoms or derivatives thereof and with serum cholesterol level reducing properties, and
b) 0 to 99.99% by weight of at least a component with serum cholesterol level effecting properties, and
c) 0 to 20% by weight of at least a pharmaceutically acceptable formulation aid.

2. The composition of claim 1, wherein the waxy acid component is a long-chain saturated or mono- or polyunsaturated hydrocarbon acid, preferably of 23 to 36 carbon atoms.

3. The composition of one of the claims 1 or 2, wherein the waxy acid component is an anhydride, a substituted or unsubstituted amid, a salt of ammonium, a salt of an alkaline or earth alkaline, a salt of an amino acid, a salt of an organic base such as a substituted ammonium, an ester of a mono-, di- or tri-alcohol, an ester of an oligo- or polyalcohol with 1 to 10 carbon atoms, an ester with a phyto-compound such as phytosterole, a sugar or a mono-, di- or triglyceride.

4. The composition of one of the claims 1 to 3, wherein the waxy acid component is of biological, geological or synthetic origin and is preferably derived from plant, animal or mineral oils and/or waxes.

5. The composition of claim 4, wherein the waxy acid component is a hydrolysed wax acid ester, that preferably contains waxy alcohols.

6. The composition of claim 5, wherein the waxy alcohols show serum cholesterol effecting properties.

7. The composition of one of the claims 1 to 6, wherein the waxy acid component acts as an additional emulsifier.

8. The composition of one of the claims 1 to 7, wherein component b) has cholesterol absorption controlling properties.

9. The composition of one of the claims 1 or 8, wherein component b) has cholesterol synthesis controlling properties.

10. The composition of one of the claims 1 to 9, wherein component b) supports cholesterol excretion.

11. The composition of one of the claims 1 or 8, wherein component b) is a sterol compound of plant origin.

12. The composition of claim 11, wherein the sterol compound is a phytosterol, phytostanol, an acceptable derivative or mixtures thereof.

13. The composition of one of the claims 11 or 12, wherein the sterol component is a branched or non-branched and/or saturated or unsaturated fatty acid ester.

14. The composition of one of the claims 1 to 13, wherein the sterol component is derived from a vegetable oil or by synthesis therefrom.

15. The composition of one of the claims 1 or 9, wherein component b) is a policosanole or statine.

16. The composition of the claims 1 or 10, wherein component b) is a polysaccharid like pectine.

17. The composition of one of the claims 1 to 16, wherein it comprises at least one ester of a phytosterol and/or a phytostanol and a fatty- or waxy acid of 23 to 40 carbon atoms.

18. The composition of one of the claims 1 to 17, wherein it comprises 1 to 40% by weight of the waxy acid component and 20 to 90% by weight of component b).

19. The composition of one of the claims 1 to 18, wherein the weight-to-weight ratio of the waxy acid component and component b) is within the range of 1 to 100.

20. The composition of one of the claims 1 to 19, wherein the formulation aid c) is one of the group consisting of fillers, stabilizers such as antioxidants, diluents, binders, buffers, lubricants and aromatics, preservatives, emulsifiers, coating agents, suspending agents and surface active compounds.

21. The composition of one of the claims 1 to 20, wherein it comprises additionally other components with serum cholesterol effecting properties such as tocotrienols, saponins, fibers, long-chain waxy alcohols of 20 to 50 carbon atoms and their acceptable derivatives, artichoke-extracts and alfalfa-extracts, statins, bile sequestrants or niacin.

22. The composition of one of the claims 1 to 21, wherein it is in a small particle form, preferably in liposome or micellar form.

23. The composition of one of the claims 1 to 22, wherein it was produced by means of natural emulsifiers such as proteins, phospholipids such as lecithin, bile acids, fatty- or waxy acid soaps, saponins or glycolipids and/or synthetic emulsifiers such as mono- or diglycerides, esters of acetic, citric, lactic and tartaric acid, stearoyllactylate and salts thereof, sugar esters and (poly-)sorbitans.

24. The composition of one of the claims 1 to 23, wherein the weight-to-weight ratio of the components a) and b) is 1:99.

25. The composition of one of the claims 1 to 24, wherein the weight-to-weight ratio of the components a), b) and c) is 1:79:20.

26. Phytosterol-waxy acid-esters, suitable as an ingredient of the composition according to the claims 1 to 25.

27. Ester of claim 26, wherein it contains as phytosterol component a branched or non-branched and/or saturated or unsaturated fatty-acid ester and/or, wherein the waxy acid component, preferably with 23 to 50 carbon atoms, is of vegetable or lignit origin.

28. Dosage formulation comprising the composition of one of the claims 1 to 25 as tablet, chewable tablet, capsule, barr, beverage, oil, oil- or fat-based emulsion, such as margarine or mayonnaise, milk-based product, such as a yogurt or a shake, or as functional food.

29. A composition for effecting serum cholesterol levels comprising
a) at least one waxy acid with 23 to 50 carbon atoms or derivatives thereof and with serum cholesterol level reducing properties, and
b) 0 to 99.99% by weight of at least a component with serum cholesterol level effecting properties, and
c) 0 to 20% by weight of a pharmaceutically acceptable formulation aid.

30. The composition of claim 29, wherein the waxy acid component is a long-chain saturated or mono- or polyunsaturated hydrocarbon acid, preferably of 23 to 36 carbon atoms.

31. The composition of one of the claim 29,wherein the waxy acid component is an anhydride, a substituted or unsubstituted amid, a salt of ammonium, a salt of an alkaline or earth alkaline, a salt of an amino acid, a salt of an organic base such as a substituted ammonium, an ester of a mono-, di- or tri-alcohol, an ester of an oligo- or polyalcohol with 1 to 10 carbon atoms, an ester with a phyto-compound such as phytosterole, a sugar or a mono-, di- or triglyceride.

32. The composition of one of the claim 29, wherein the waxy acid component is of biological, geological or synthetic origin and is preferably derived from plant, animal or mineral oils and/or waxes.

33. The composition of claim 32, wherein the waxy acid component is a hydrolysed wax acid ester, that preferably contains waxy alcohols.

34. The composition of claim 33, wherein the waxy alcohols show serum cholesterol effecting properties.

35. The composition of one of the claim 29, wherein the waxy acid component acts as an additional emulsifier.

36. The composition of one of the claim 29, wherein component b) has cholesterol absorption controlling properties.

37. The composition of one of the claim 29, wherein component b) has cholesterol synthesis controlling properties.

38. The composition of one of the claim 29, wherein component b) supports cholesterol excretion.

39. The composition of one of the claim 29, wherein component b) is a sterol compound of plant origin.

40. The composition of claim 39, wherein the sterol compound is a phytosterol, phytostanol, an acceptable derivative or mixtures thereof.

41. The composition of one of the claim 39, wherein the sterol component is a branched or non-branched and/or saturated or unsaturated fatty acid ester.

42. The composition of one of the claim 29, wherein the sterol component is derived from a vegetable oil or by synthesis therefrom.

43. The composition of one of the claim 29, wherein component b) is a policosanole or statine.

44. The composition of the claim 29, wherein component b) is a polysaccharid like pectine.

45. The composition of one of the claim 29, wherein it comprises at least one ester of a phytosterol and/or a phytostanol and a fatty- or waxy acid of 23 to 40 carbon atoms.

46. The composition of one of the claim 29, wherein it comprises 1 to 40% by weight of the waxy acid component and 20 to 90% by weight of component b).

47. The composition of one of the claim 29, wherein the weight-to-weight ratio of the waxy acid component and component b) is within the range of 1 to 100.

48. The composition of one of the claim 29, wherein the formulation aid c) is one of the group consisting of fillers, stabilizers such as antioxidants, diluents, binders, buffers, lubricants and aromatics, preservatives, emulsifiers, coating agents, suspending agents and surface active compounds.

49. The composition of one of the claim 29, wherein it comprises additionally other components with serum cholesterol effecting properties such as tocotrienols, saponins, fibers, long-chain waxy alcohols of 20 to 50 carbon atoms and their acceptable derivatives, artichoke-extracts and alfalfa-extracts, statins, bile sequestrants or niacin.

50. The composition of one of the claim 29, wherein it is in a small particle form, preferably in liposome or micellar form.

51. The composition of one of the claim 29, wherein it was produced by means of natural emulsifiers such as proteins, phospholipids such as lecithin, bile acids, fatty- or waxy acid soaps, saponins or glycolipids and/or synthetic emulsifiers such as mono- or diglycerides, esters of acetic, citric, lactic and tartaric acid, stearoyllactylate and salts thereof, sugar esters and (poly-)sorbitans.

52. The composition of one of the claim 29, wherein the weight-to-weight ratio of the components a) and b) is 1:99.

53. The composition of one of the claim 29, wherein the weight-to-weight ratio of the components a), b) and c) is 1:79:20.

54. Phytosterol-waxy acid-esters, suitable as an ingredient of the composition according to the claim 29.

55. Ester of claim 54, wherein it contains as phytosterol component a branched or non-branched and/or saturated or unsaturated fatty-acid ester and/or, wherein the waxy acid component, preferably with 23 to 50 carbon atoms, is of vegetable or lignit origin.

56. Dosage formulation comprising the composition of claim 29, as tablet, chewable tablet, capsule, barr, beverage, oil, oil- or fat-based emulsion, such as margarine or mayonnaise, milk-based product, such as a yogurt or a shake, or as functional food.
